(19) United States
(12) Patent Application Publication
(10) Pub. No.: US 2023/0125377 A1
(43) Pub. Date: Apr. 27, 2023

(54) LABEL-FREE REAL-TIME HYPERSPECTRAL ENDOSCOPY FOR MOLECULAR-GUIDED CANCER SURGERY

(71) Applicants: THE REGENTS OF THE UNIVERSITY OF CALIFORNIA, Oakland, CA (US); BOARD OF REGENTS, THE UNIVERSITY OF TEXAS SYSTEM, Austin, TX (US)

(72) Inventors: Liang Gao, Santa Clara, CA (US); Baowei Fei, Richardson, TX (US)

(73) Assignees: THE REGENTS OF THE UNIVERSITY OF CALIFORNIA, Oakland, CA (US); BOARD OF REGENTS, THE UNIVERSITY OF TEXAS SYSTEM, Austin, TX (US)

(21) Appl. No.: 18/045,969

(22) Filed: Oct. 12, 2022

Related U.S. Application Data
(63) Continuation of application No. PCT/US2021/031347, filed on May 7, 2021.
(60) Provisional application No. 63/022,272, filed on May 8, 2020.

(51) Int. Cl. G01J 3/28; G01J 3/02

(52) U.S. Cl. CPC G01J 3/2823; G01J 3/0229; A61B 5/0075

(57) ABSTRACT

Systems and methods are provided for label-free, real-time hyperspectral imaging (HSI) endoscopy for molecular-guided surgery of cancers without the need for an exogenous contrast agent. One device is a high-speed image mapping spectrometer integrated with a white-light reflectance fiberoptic bronchoscope. The imaging system has a parallel acquisition instrument that captures a hyperspectral datacube that may be pre-processed and features extracted and a discriminative feature set is selected and used for the classification of cancer and benign tissue. An algorithm that enables fast and accurate tissue classification may also be applied that utilizes a supervised deep-learning-based framework that is trained with the clinically visible tumor and benign tissue during surgery and then applied to identify the residual tumor.

### CROSS-REFERENCE TO RELATED APPLICATIONS

This application claims priority to, and is a 35 U.S.C. § 111(a) continuation of, PCT international application number PCT/US2021/031347 filed on May 7, 2021, incorporated herein by reference in its entirety, which claims priority to, and the benefit of, U.S. provisional patent application Ser. No. 63/022,272 filed on May 8, 2020, incorporated herein by reference in its entirety. Priority is claimed to each of the foregoing applications.

The above-referenced PCT international application was published as PCT International Publication No. WO 2021/226493 A1 on Nov. 11, 2021, which publication is incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to imaging and surgical instrumentation systems and methods and more particularly to a hyperspectral imaging surgical endoscope instrument and methods for real-time intraoperative tumor margin assessment in vivo and in situ.

### 2. Background

Lung cancer is the second most common cancer found in both men and women, and it accounts for 25% of all cancer deaths, resulting in over 1.4 million deaths worldwide per year. Despite advances in therapy, the 5-year survival rate for lung cancer is approximately 16%, which is the lowest among all common cancers. Currently, surgery remains the primary therapeutic method for non-small cell lung cancer and over 70% of stage I and II non-small cell lung cancer patients undergo surgery. The most important predictor of patient survival for almost all cancers is complete surgical resection of the primary tumor. Currently, however, over 40% of patients that undergo surgery leave the operating room without a complete resection due to missed cancerous lesions. Because the tumor margin status influences local recurrence and long-term survival, it is crucial for the surgeon to identify tumor margins accurately and excise all cancerous tissues with negative margins. Surgical resection margins, therefore, are a key quality metric for the surgical management of non-small cell lung cancer.

Conventional intraoperative margin assessment of tumors relies on visual inspection and palpation by the surgeon. The necessary reliance on subjective judgment frequently jeopardizes the accuracy of the surgical resection. Although the excised tissue specimens may be further evaluated by frozen section analysis, the process is time-consuming, and the results are often inconclusive.

Despite advances in preoperative imaging such as CT and MRI, the surgery itself is still primarily guided by the ability of the surgeon to identify the lesion and make contemporary judgments on its margins with light endoscopy in the operating room.

The use of autofluorescence imaging (AFI) and fluorescence imaging (FI) are standard-of-care endoscopic techniques for imaging tumor-specific contrast to help guide surgeons during surgery. Although AFI techniques image endogenous chromophores, the process suffers from low sensitivity and specificity in assessing tumor margins.

In contrast, FI labels tumors with exogenous fluorophores, leading to a significantly improved classification accuracy. While the FI approach significantly improves the accuracy of tumor identification, it faces significant regulatory challenges since the number of FDA-approved fluorescent dyes are very limited.

Another major weakness of FI is the over-reliance of preclinical testing in tumor cell lines that are monolithically positive for the molecular target of interest. For instance, when a receptor-targeted probe is being tested, it is normally tested on a tumor line that has exceptionally high expression of that receptor. However, when it is deployed in human trials, the range of tumors imaged can be significantly more variable than the cell line that was originally tested on. Additionally, as tumors grow, the phenotypic characteristics can vary throughout the tumor in terms of gene expression, protein expression, and mutated protein expression, altering the expression level of fluorescence probes and confounding the interpretation of observed contrast.

Furthermore, the expression of a molecular-specific dye in patients is rather heterogeneous, and the expression can vary over time and with sample handling. Therefore, the interpretation of fluorescence results can be easily confounded by uncertain or inconclusive data about the observed target contrast.

Therefore, there is a need for new endoscopy methods that provide high sensitivity and do not require the use of fluorescent labeling and is capable of supporting critical decision-making in the operating room.

### BRIEF SUMMARY

Systems and methods are provided for label-free, real-time hyperspectral imaging endoscopy for molecular guided surgery. The methods demonstrate high sensitivity and specificity for tumor detection without the use of fluorescent labeling. The methods generally combine snapshot hyperspectral imaging and machine learning to implement a real-time data acquisition. Furthermore, the methods can be applied to standard clinical practice since they require minimal modification to the established white-light surgical imaging procedures known in the art. The methods can extend a surgeon's vision at both the cellular and tissue levels to improve the ability of the surgeon to identify the lesion and its margins.

The present technology is facilitated by a snapshot Hyperspectral Imaging (HSI) technique, Image Mapping Spectrometry (IMS), and the development of a machine-learning-based HSI processing pipeline. The synergistic integration of advanced instrumentation and algorithms makes the technology presented herein uniquely positioned in addressing the leading challenges in molecular-guided surgery of cancer.

The overall rationale of using HSI for molecular-guided imaging is that the tissue's endogenous optical properties such as absorption and scattering change during the progression of the disease and the spectrum of light remitted from the tissue carries quantitative diagnostic information about tissue pathology. The molecular-guided surgery provides a more accurate visualization of tumor margins through imaging either endogenous chromophores, such as reduced nicotinamide adenine dinucleotide (NADH), flavin adenine dinucleotide (FAD), and porphyrins, or with exogenous fluorophores, such as indocyanine green (ICG) and methylene blue. Compared with standard unaided vision using white light imaging, the molecular-imaging surgical cameras provided herein not only allow more complete resections but also improve safety by avoiding unnecessary damage to normal tissue.

Conventional intensity-based cameras measure only the two-dimensional spatial distribution of light. In contrast, HSI captures light in three dimensions, acquiring both the spatial coordinates (x, y) and wavelengths (L) of the incident photons simultaneously. The obtained information can be used to facilitate a variety of surgical operations, such as identifying lesions, localizing nerves, and monitoring tissue perfusion.

However, despite being a powerful tool, conventional HSI devices face a major challenge in real-time data acquisition. To acquire a spectral datacube (x, y, λ), they scan in the either spatial domain or spectral domain, a fact that causes a severe trade-off problem between the number of photons the instrument can collect and the frame acquisition rate. The scanning mechanism thus limits the utility of these hyperspectral imagers in real-time imaging applications. Particularly in surgery, because of the movement of the probe or patient, the slow acquisition speed would result in severe motion artifacts. Therefore, to apply HSI in molecular-guided surgery, the hyperspectral datacube must be acquired in a snapshot format.

Compared with autofluorescence imaging (AFI) and fluorescence imaging (FI), HIS also has a unique advantage in fitting into the standard clinical practice because it requires minimal modification to the existing white-light surgical imaging procedure. Only a simple replacement of the original intensity-based camera with an HSI device is needed. The resultant method can seamlessly blend into the current surgical workflow while providing an immediate clinical goal with important new information that affects the patient outcome.

The technology provides a real-time hyperspectral imaging surgical endoscope based in part on imaging mapping spectrometry. In one embodiment, a high-resolution, high-speed image mapping spectrometer is integrated with a white-light reflectance fiberoptic bronchoscope. This probe is a real-time hyperspectral imaging surgical endoscope that can simultaneously capture 100 spectral channel images in the visible wavelengths (400-900 nm) within a 120° field of view. The frame rate is limited by only the readout speed of the camera, which may be up to 50 Hz, allowing real-time image acquisition and data streaming.

The acquired HSI data is preferably pre-processed by spectral normalization, image registration, glare detection, and curvature correction. Image features are then extracted from the HSI data, and a discriminative feature set will be selected and used for the classification of cancer and benign tissue. At the same time, the developed convolutional neural networks (CNN) are used to automate the real-time hyperspectral image processing. Overall, HSI can extend a surgeon's vision at both the cellular and tissue levels, improving the surgeon's ability to identify the lesion and make judgments on its margins and thereby significantly increase the success rate of the surgery.

Accordingly, aspects of the presented technology include advanced label-free hyperspectral imaging instrumentation and a machine-learning-based algorithm for real-time intraoperative tumor margin assessment in vivo and in situ. The technology is broadly applicable to many types of tumors, including lung cancer which is one of the most aggressive human malignancies that affect both men and women worldwide.

According to one aspect of the technology, a high-resolution, high-speed imaging mapping spectrometer device and quantification tools are provided that are applicable to detecting cancer.

In one embodiment, an imaging device is provided that comprises a real-time hyperspectral imaging surgical endoscope based on imaging mapping spectrometry.

In another embodiment, a high-resolution, a high-speed image mapping spectrometer is integrated with a white-light reflectance fiberoptic bronchoscope.

In one embodiment, the resultant probe can simultaneously capture about 100 spectral channel images in the visible wavelengths (about 400 nm to about 900 nm) within about a 120° field of view.

In another embodiment, the frame rate, limited by only the readout speed of the camera, is up to about 50 Hz, allowing for real-time image acquisition and data streaming.

In various embodiments, the technology provides image quantification methods and deep convolutional neural networks (CNN) for real-time hyperspectral image processing.

In one embodiment, HSI data is pre-processed by spectral normalization, image registration, glare detection, and curvature correction.

In another embodiment, image features are extracted from the HSI data, and a discriminative feature set is selected and used for the classification of cancer and benign tissue.

In various embodiments, CNNs are used to automate the real-time hyperspectral image processing are provided.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a schematic system diagram with optical schematic of the snapshot hyperspectral endoscope with GRIN and gradient index according to one embodiment of the technology.

FIG. 2 is an optical schematic diagram of a combination of multiple low-resolution IMSs through beam splitting according to an alternative embodiment of the technology.

FIG. 3 is a functional process flow diagram showing operating principles of image mapping spectrometry.

FIG. 4 is a functional block diagram of quantitative HSI image processing according to one embodiment of the technology.

FIG. 5 is a functional flow diagram of the data processing and deep learning architecture of the apparatus and methods.

### DETAILED DESCRIPTION

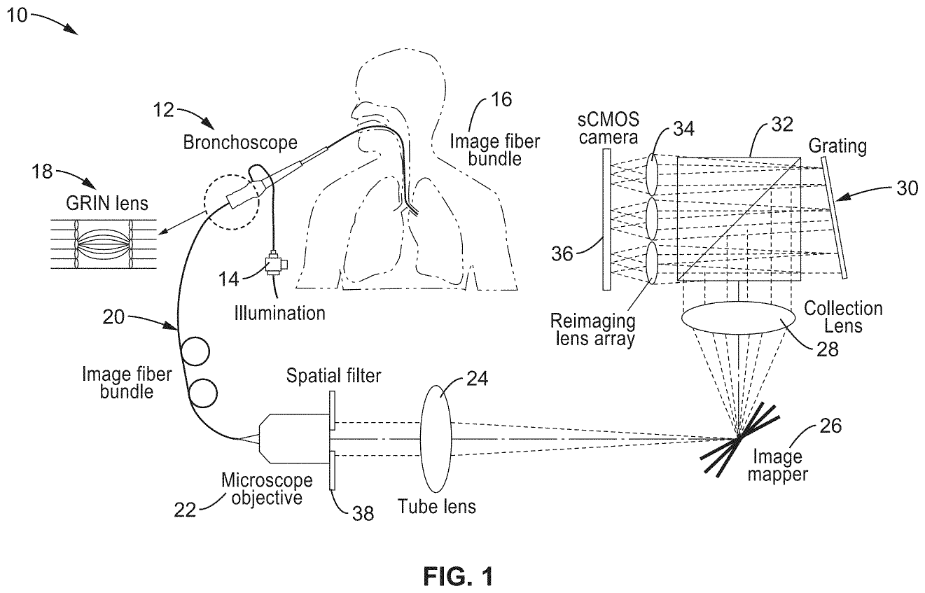

Referring more specifically to the drawings, for illustrative purposes, systems and methods for label-free, real-time hyperspectral imaging endoscopy for molecular guided surgery are generally shown. Several embodiments of the technology are described generally in FIG. 1 to FIG. 5 to illustrate the characteristics and functionality of the devices, systems and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

The illustrated real-time HSI surgical endoscopy apparatus is an intraoperative imaging modality that can provide real-time, label-free tumor margin assessment with high accuracy. Compared to known imaging methods in the art, the real-time HSI surgical endoscope features three important technological innovations. First, the integration of a snapshot hyperspectral imager with a fiberoptic bronchoscope enables a “spectral biopsy” of pulmonary lesions in vivo and in situ. Because the imaging techniques are based on white-light reflectance requiring no exogenous contrast agents, they can be readily fitted into current surgical workflows, accelerating its clinical translation.

Second, the HSI classification algorithms and quantification tools, which are built on machine learning algorithms, are particularly suited for cancer detection and surgical margin assessment.

Third, the in-vivo animal and ex-vivo surgical tissue imaging will generate the first public HSI database for label-free tumor classification, providing a testbed for data training and validation and thereby facilitating the development of new machine-learning-based algorithms specifically tailored to various tumor types.

Turning now to FIG. 1, an embodiment of the optical configuration of a snapshot hyperspectral endoscopy system 10 is shown schematically. The system 10 generally integrates a high-resolution, high-speed Image Mapping Spectrometry (IMS) process with a white-light fiberoptic bronchoscope 12, enabling hyperspectral imaging of pulmonary lesions in vivo and in situ. Because the method images endogenous chromophores, it requires neither fluorescence labeling nor specialized filter sets, facilitating its integration into the current surgical workflow.

For surgical compatibility, one adaptation of the system is based on an interventional fiberoptic bronchoscope 12, which has a large instrument channel for biopsy and electrosurgery. In this illustration, a light source 14 such as a broadband Xenon light source, is coupled to the illumination channel of the bronchoscope 12, to illuminate an imaging site through an integrated light guide and facilitating insertion to a desired location.

The reflected light from the target is then collected by an imaging lens at the distal end of the probe, which is transmitted through an image fiber bundle 16 (e.g. ˜3k fibers; bundle diameter, 0.7 mm; fiber core diameter, 10 μm), and forms an intermediate image at the proximal end of the bronchoscope 12 (dashed circle).

To pass this image to the IMS, in one embodiment, a gradient-index (GRIN) lens 18 (e.g. 1:1 relay; length, 0.5 pitch; GRINTECH) is coupled to the bronchoscope imaging lens on one end and to a second image fiber bundle 20 (e.g. bundle diameter, 0.7 mm; fiber core diameter, 5 μm; Schott) on the other.

In the embodiment shown in FIG. 1, the output image (diameter, 0.7 mm) is then magnified by a 4f imaging system preferably comprising a microscope objective 22 (e.g. Olympus PLN 20×) and a tube lens 24 (focal length, 180 mm) and then relayed to an image mapper 26 in the IMS. In this embodiment, an optional spatial filter 38 is positioned at the back aperture of the objective lens 22 to remove the obscuration pattern of the fiber bundle 20.

In one preferred embodiment, the image mapper 26 comprises 150 total facets, each 100 μm wide and 15 mm in length. In this illustration, the mapper 26 comprises a total of 100 assorted 2D tilt angles (a combination of nine x tilts and nine y tilts), enabling hyperspectral imaging of 100 spectral bands. The preferred parameters of the image mapper 26 are 150 mirror facets; Mirror facet length 15 mm; Mirror facet width 100 μm; Mirror facet x tilts±N×0.011+0.0055 radians (N=1:5) and Mirror facet y tilts±M×0.011+0.0055 radians (M=1:5).

At the image mapper 26, in this illustration, the imaged PSF of the fiber is matched to the width of mirror facet width, resulting in an effective NA of 0.0025. In one embodiment the light rays reflected from different mirror facets are collected by a collection objective lens 28 (e.g. NA, 0.25; focal length, 90 mm; Olympus MVX PLAPO1×) and enter corresponding pupils (not shown) at the back aperture of the lens 28. In one embodiment, the angular separation distance between adjacent pupils is 0.022 radians, which is greater than twice of the NA (0.005) at the image mapper, thereby eliminating the crosstalk between pupils.

In one embodiment the light from the image mapper 26 is then spectrally dispersed by a ruled diffraction grating 30 (e.g. 220 grooves/mm; blaze wavelength, 650 nm; Littrow configuration; 80% efficiency; Optometrics) and splitter 32 (e.g. dichroic mirror) and then reimaged by an array of lenslets 34 (e.g. 10×10; focal length, 10 mm; diameter, 2 mm). Although a diffraction grating is preferred, a diffracting prism can also be used.

In one embodiment, the resultant image from the lenslets 34 is measured by a large format, high sensitivity sCMOS camera 36 (e.g. 2048×2048 pixels; pixel size, 11 μm; KURO, Princeton Instruments) within a single exposure. Since the magnification from the image mapper 26 to the detector array is 0.11, in this illustration, the image associated with each lenslet of the array 34 is 1.65×1.65 mm² in size, sampled by 150×150 camera pixels of camera 36. In one embodiment, the spacing created for spectral dispersion between two adjacent image slices is about 1.1 mm and is sampled by 100 camera pixels. Given 500 nm spectral bandwidth, the resultant spectral resolution is approximately 5 nm.

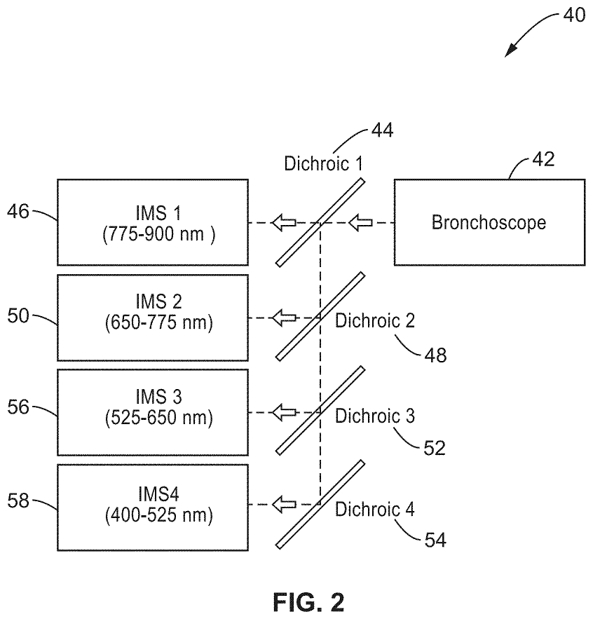

Alternatively, a system 40 with multiple low-spectral-sampling IMSs can be used, each IMS measuring a separate spectral range. As shown schematically in FIG. 2, several duplicated low-spectral-sampling IMS elements can be employed replacing their spectral dispersion units with diffraction gratings. Next, their optical paths are combined using dichroic filters with a descending order of their cut-off wavelengths.

In the illustration of FIG. 2, the image from the bronchoscope 42 is directed to a dichroic mirror 44 and through to the IMS 46. The IMS 46 has a wavelength range of (775-900) in this case. The split beam also goes to a second dichroic mirror 48 and second IMS 50. The beam also goes through subsequent dichroic mirrors 52, 54 and subsequent IMS detectors 56, 58. It can be seen in the illustration of FIG. 2 that each IMS provides 24 spectral samplings in the correspondent spectral band, allowing a total of 96 spectral channels in the total wavelength range of 400 nm to 900 nm.

The resultant system 40 will have a similar spectral resolution (5.2 nm) as that offered by the high-spectral-sampling IMS shown in FIG. 1. The resultant probe in this illustration is able to simultaneously acquire 100 spectral channels in the range 400 nm to 900 nm, where visible and NIR light provides complementary information for diagnosis.

The apparatus is then preferably calibrated with a two-step calibration procedure. Calibration establishes a correspondence between each voxel in the hyperspectral datacube (x, y, λ) and a pixel location on the sCMOS camera (u, v) in the IMS. In one embodiment the complete calibration procedure comprises two steps: (1) remapping with the transformation lookup table (x, y, λ)=T⁻¹ [(u, v)], and (2) flat-field correction and spectral sensitivity correction.

Step 1: The goal of this step is to determine (x, y, λ)=T⁻¹ [(u, v)], where T⁻¹ is effectively a lookup table that is the same size as the datacube, which contains a subpixel detector value at each index. To determine T⁻¹, the forward mapping T can be computed first by sequentially illuminating integer coordinates (x, y, λ) throughout the datacube while analyzing the detector (u, v) response. Once the relationship T from the scene to the detector is established, a reverse mapping T⁻¹ or “remapping” can be applied to transform the raw detector data into a datacube.

This procedure can be accomplished by scanning a pinhole throughout the FOV of the bronchoscope at (x, y, λ) object coordinates. At each scanning location, each pinhole is sequentially illuminated with monochromatic light from about 400 nm to about 900 nm in 5 nm steps using a liquid crystal tunable filter. Each position of the pinhole provides a point image in a region on the detector in this example. The subpixel center position (u, v) of the point image can be determined with a peak-finding algorithm. Remapping the (x, y, λ) datacube using the lookup table may also be implemented in real-time using bicubic interpolation of raw detector data.

Step 2: A flat-field correction is then preferably performed to compensate for the intensity variations of mirror facet images and spectral responses of the instrument. For example, a uniform light field from an integration light sphere (e.g. Ocean Optics FOIS-1) illuminated with a radiometric standard lamp (e.g. Ocean Optics HL-3-P-CAL) can be imaged and the hyperspectral datacube recorded. Dividing all subsequent datacubes acquired by the IMS by this reference datacube will normalize the intensity response of every datacube voxel. Next, to correct for the spectral sensitivity the normalized voxel values at each spectral layer may then be multiplied with the correspondent absolute irradiance of the light source at that wavelength.

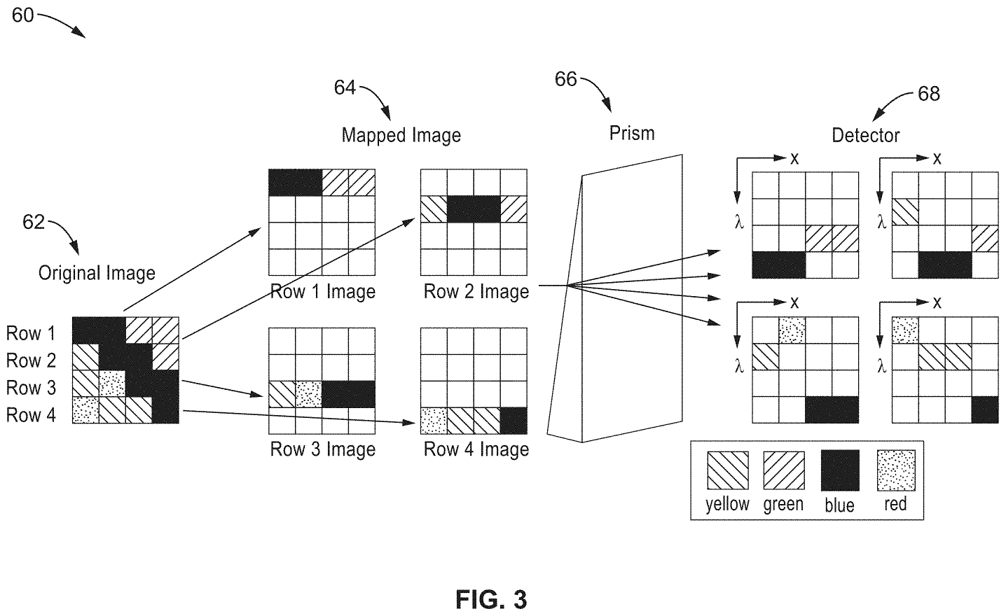

A core feature of real-time HSI surgical endoscopy is a snapshot hyperspectral imager and image mapping spectrometry (IMS). The operating principles of image mapping spectrometry are shown schematically in FIG. 3. The IMS features replace the camera in a digital imaging system, allowing one to add high-speed snapshot spectrum acquisition capabilities to a variety of imaging modalities such as microscopy, macroscopy, and ophalmoscopy to maximize the collection speed.

The IMS process addresses the high temporal resolution requirements found in time-resolved multiplexed biomedical imaging. Conventional spectral imaging devices acquire data through scanning, either in the spatial domain (as in confocal laser scanning microscopes) or in the spectral domain (as in filtered cameras). Because scanning instruments cannot collect light from all elements of the dataset in parallel, there is a loss of light throughput by a factor of Nₓ×N_{y} when performing scanning in the spatial domain over Nₓ×N_{y} spatial locations, or by a factor of N_{λ} when carrying out scanning in the spectral domain measuring N_{λ} spectral channels.

In the embodiment shown in FIG. 1, the IMS is a parallel acquisition instrument that captures a hyperspectral datacube without scanning. It also allows full light throughput across the whole spectral collection range due to its snapshot operating format. The IMS uses a designed mirror, termed an image mapper 26, that has multiple angled facets to redirect portions of an image to different regions on a detector array 36.

In the embodiment of the methods 60 shown in FIG. 3, the original image 62 is mapped to produce mapped image slices 64. By redirecting slices of the image so that there is space between slices on the detector array 68, a prism 66 or diffraction grating 26 can be used to spectrally disperse light in the direction orthogonal to the length of the image slice. In this way, with a single frame acquisition from the camera, a spectrum from each spatial location in the image can be obtained. The original image 62 can be reconstructed by a simple remapping of the pixel information.

To reflect the image zones into different directions, individual facets of the image mapper 26 have different tilt angles with respect to the two axes in the plane of the slicer. This mapping method establishes a fixed one-to-one correspondence between each voxel in the datacube (x, y, λ) (x, y, spatial coordinates; λ, wavelength) and each pixel on the camera 68. The position-encoded pattern on the camera simultaneously provides the spatial and spectral information within the image. Since the acquired data results directly from the object's irradiance, no reconstruction algorithm is required, and simple image remapping produces the image and data displays.

It can be seen that the HSI methods 60 acquires a stack of two-dimensional images over a wide range of spectral bands and generates a three-dimensional hyperspectral datacube containing rich spectral-spatial information. The resultant hyperspectral datacubes are generally large in size. The primary challenge of hyperspectral datacube analysis, therefore, lies in real-time processing of these large spectra-spatial datasets and rendering the images of diagnostic importance.

To address the large amounts of hyperspectral data and the need for fast processing, the methods preferably extract and select features that “optimally” characterize the difference between cancer and benign tissue, thereby significantly reducing the dimension of HSI dataset. An algorithm that enables fast and accurate tissue classification is also applied that utilizes a supervised deep-learning-based framework that is trained with the clinically visible tumor and benign tissue during surgery and then applied to identify the residual tumor.

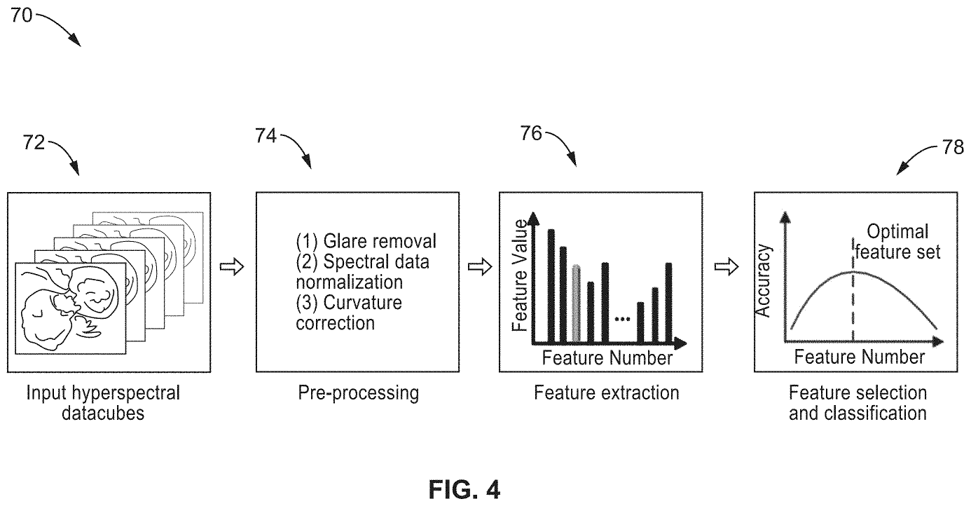

One embodiment of a post HSI data process 70 is shown in FIG. 4. The HSI data acquired at block 72 is pre-processed at block 74 and then features are extracted at block 76. The extracted features are then selected and classified at block 78 of FIG. 4.

A variety of pre-processing schemes are available for preprocessing of HSI images at block 74 and extracting features at block 76. In one embodiment, the preprocessing 74 of HSI images comprises three phases: (1) Glare removal; (2) Spectral data normalization and (3) Curvature correction.

The optical endoscopic images that were acquired during surgery are often strongly affected by glare artifacts, which present a major problem for surgical image analysis. In HSI, glare alters the intensities of the pixels in each spectral band and consequently changes the spectral fingerprint, which could, in turn, introduce artifacts in feature extraction 76 and hence deteriorate classification 78 results.

Because glare pixels generally have a higher total reflectance than normal pixels, in one embodiment, the glare pixels are detected and removed in two steps: 1) calculate the total reflectance of each pixel by summing the voxels of a hyperspectral cube along the wavelength axis, and 2) compute the intensity histogram of this image, fit the histogram with a log-logistic distribution, and then experimentally identify a threshold that separates glare and nonglare pixels. The hyperspectral data associated with glare pixels are excluded from the analysis.

The purpose of spectral data normalization is to remove the spectral nonuniformity of the illumination light source (e.g. Xenon) and the influence of the dark current of the detector. In one embodiment, the distal end of the probe is inserted into an integration light sphere (Ocean Optics FOIS-1) and illuminated with the Xenon light through the integrated light guide to capture a baseline hyperspectral datacube Iₓ. Next, the light is turned off and a dark frame I_{D} is captured using the same exposure time. Next the IMS measurement is normalized as I_{N}=[I_{R}−I_{D}]/[I_{X}−I_{D}], where I_{R} is the raw hyperspectral datacube.

The curvature correction processing compensates for spectral variations caused by the elevation of tissue. At the time of imaging, tumors generally protrude outside of the skin, and are therefore closer to the detector than the normal skin around it. A further normalization may need to be applied to compensate for differences in the intensity of light recorded by the camera due to the elevation of tumor tissue. The light intensity changes can be viewed as a function of the distance and the angle between the surface and the detector. Two spectra of the same point acquired at two different distances and/or angles will have the same shape but will vary by a constant. By dividing each individual spectrum by a constant calculated as the total reflectance at a given wavelength A will remove the distance and angle dependence as well as dependence on an overall magnitude of the spectrum. This normalization step ensures that variations in reflectance spectra are only a function of wavelength, and therefore the differences between cancerous and normal tissue are not affected by the elevation of tumors.

Spectral features that are extracted at block 76 may include: (1) first-order derivatives of each spectral curve, which reflect the variations of spectral information across the wavelength range; (2) second-order derivatives of each spectral curve, which reflect the concavity of the spectral curve; (3) mean, std, and total reflectance at each pixel, which summarize the statistical characteristics of the spectral fingerprint; and (4) Fourier coefficients (FCs). Each feature is standardized to its z-score by subtracting the mean from each feature and then dividing by its standard deviation. The metrics initially increased with the number of features, reached a maximum, and then decreased as the feature set went to its maximum size.

Because the method can differentiate tumor and normal tissue in vivo without administering contrast agents to humans, it can be readily integrated into current surgical workflow schemes, thereby providing immediate health benefits to patients.

The methods allow the surgeon to accurately localize and resect lung tumors while preserving healthy lung function. Accurate and contemporary classification capabilities have the potential to make a major impact in reducing the local and regional recurrence rates of lung cancer after surgery and improving the overall patient survival rate.

Moreover, beyond lung cancer, the real-time HSI endoscopy system can also be used for imaging other malignant lesions, such as brain cancer, oral cancer, and colon cancer. Like lung cancer, their progression is often accompanied by abnormal structural and molecular changes, which can be inferred from HSI measurement. Delineating the tumor margins based on the spectral signatures can dramatically improve the safety and accuracy of surgical resection in these cancers as well.

After feature extraction, the feature dimension will increase to several hundreds or thousands. Such a high dimension poses significant challenges to HSI classification. Feature selection finds a feature set s with n wavelengths λᵢ, which “optimally” characterize the difference between cancer and benign tissue. To achieve this “optimal” condition, a maximal relevance and minimal redundancy (mRMR) framework is preferably applied to maximize the dependency of each spectral feature on the target class labels and to minimize the redundancy among individual features simultaneously. Relevance is characterized by mutual information I(x; y), which measures the level of similarity between two random variables x and y:

$$I ( x ; y ) = \int \int p ( x , y ) log \frac{p ( x , y )}{p ( x ) p ( y )} dxdy$$

where p(x, y) is the joint probability distribution function of x and y, and p(x) and p(y) are the marginal probability distribution functions of x and y, respectively.

Each pixel with M features Λ={λᵢ, i=1, . . . M}, M=904 is represented, and the class label (tumor or normal) with c. Then the maximal relevance condition is to search features, which maximize the mean value of all mutual information values between individual features λᵢ and class c:

$$max D ( s , c ) , D = \frac{1}{\vert S \vert} {\sum }_{{λ}_{i} ∈ S} I ( {λ}_{i} , c )$$

The features selected by the maximal relevance condition are likely to have redundancy, which means that the dependency among these features could be large. When two features highly depend on each other, the respective class-discriminative power would not change much if one of them were removed. So the minimal redundancy condition can be added to select mutually exclusive features:

$$min R ( s ) , R = \frac{1}{{\vert S \vert}^{2}} {\sum }_{{λ}_{i} , {λ}_{j} ∈ S} I ( {λ}_{i} , {λ}_{j} )$$

The simple combination of these two conditions forms the criterion “minimal-redundancy-maximal-relevance” (mRMR), which can optimize D and R simultaneously:

max Φ(D,R),Φ=D−R

In practice, incremental search methods can be used to find the near-optimal features defined by Φ(⋅). Suppose a feature set Sₘ₋₁ with m−1 features has already been identified. The task is to select the mth feature from the set {Λ−Sₘ₋₁}. This may be done by selecting the feature that maximizes Φ(⋅). The respective incremental algorithm optimizes the following condition:

$${max}_{{λ}_{j ∈} Λ - {S}_{m - 1}} [ I ( {λ}_{j} ; c ) - \frac{1}{m - 1} {\sum }_{{λ}_{i} ∈ {S}_{m - 1}} I ( {λ}_{j} ; {λ}_{i} ) ]$$

To extract the maximum diagnostic information that can be used to differentiate the tumor from surrounding normal tissue, a deep-learning-based framework may be used for hyperspectral image processing and quantification, which includes image preprocessing, feature extraction and selection, and image classification results. The supervised deep-learning-based framework is trained with the clinically visible tumor and benign tissue during surgery and then applied to identify the residual tumor. The resultant algorithm enables fast and accurate tissue classification.

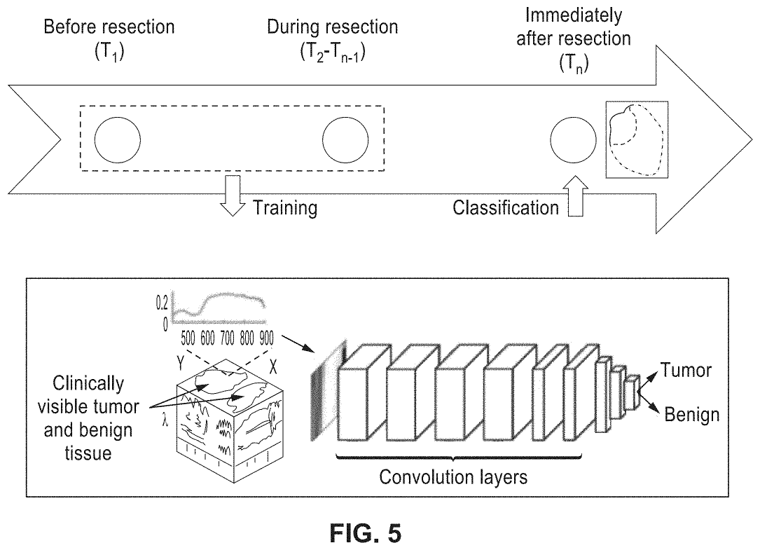

A flowchart of data processing and deep learning architecture is shown schematically in FIG. 5 for a CNN with prior knowledge of a specific cancer. For surgical applications, HSI data can be acquired at different time points: i) immediately after the tumor is surgically exposed but before resection (T₁), ii) during resection (T₂-Tₙ₋₁), and iii) immediately after resection (Tₙ) as seen in the top section of FIG. 5. On T₁-Tₙ₋₁ images, the surgeon can mark regions of interest (ROIs) that show clinically visible cancer or benign tissue and then use them as references. This prior knowledge is used as input to the deep learning algorithms for identifying residual tumor on the Tₙ image.

The spectral and spatial information is then combined to construct spectral-spatial features for each pixel. The neighboring region of a center pixel will include eight rays in a 45-degree interval. The pixels along the ray are extended around the pixel with a radius (e.g., 10 pixels). The pixels are flattened along the ray into one vector, and this is used as the spatial feature of the center pixel. As each pixel along the ray also has many spectral bands, all of the bands are further flattened into one long vector.

The resultant spatial-spectral dataset is input into the deep supervised learning algorithm, and latent representations can be learned using stacked auto-encoders (SAE). To integrate the layers of neural networks and perform tumor classification based on the feature learned, the algorithm tunes the whole network with a multinomial logistic regression classifier. Backpropagation can be used to adjust the network weights in an end-to-end fashion.

In one embodiment, tumor tissue classification is evaluated by gold-standard histologic maps. There are two types of tissue (benign & tumor) used as the training dataset. The testing dataset from the specimens that have mixed tumor and benign tissue. The deep learning method is applied to classify the benign and tumor tissue. With the registered histologic images, it is possible to evaluate the deep learning classification pixel by pixel and to calculate the classification accuracy.

The technology described herein may be better understood with reference to the accompanying examples, which are intended for purposes of illustration only and should not be construed as in any sense limiting the scope of the technology described herein as defined in the claims appended hereto.

### Example 1

System design constraints and parameters were evaluated to guide the fabrication and testing of the system. Because the imaging is by a fiberscope at the front end, the spatial resolution is fundamentally limited by the number of fibers (typically ˜3k) that are in the bundle.

Given hexagonal fiber arrangement sampling a 120° field of view (FOV), the spatial resolution was approximately 2° at the object side. When this image is passed to the IMS, the spatial samplings at the mapper are Nₓ=N_{y}=150, satisfying the Nyquist sampling condition. Measured by the IMS, the corresponding hyperspectral datacube voxels are one-to-one mapped to the sCMOS camera pixels. Therefore, the product of spatial and spectral samplings (Nₓ×N_{y}×N_{A}) cannot exceed the number of camera pixels (Nᵤ×Nᵥ). A detector utilization factor η was defined as the ratio of these two physical quantities, i.e., η=Nₓ×N_{y}×N_{λ}/(Nᵤ×Nᵥ), and its ideal value is one.

In one embodiment, the image associated with each lenslet was 1.65×1.65 mm² in size, while the total detector area allocated to each lenslet was 2×2 mm². Peripheral void spaces were created to avoid the crosstalk between adjacent lenslet images to tolerate fabrication errors, resulting in a detector utilization factor η=0.53. Lastly, because the instrument acquires a hyperspectral datacube in a snapshot, the frame rate was limited by only the readout speed of the sCMOS camera and was up to 50 Hz. Accordingly, the designed system parameters included a FOV of 120°, a spatial resolution of 2°, a spectral range of 400-900 nm, a spectral resolution of 5 nm and a frame rate of up to 50 Hz.

The light throughput of the system is determined by the throughputs of both front optics (i.e., the fiber bronchoscope) and the IMS and the quantum yield of the detector. The throughput of the fiber bronchoscope, η_{B}, is primarily limited by the fiber coupling efficiency, and it is typically 50%. The throughput of the IMS, η_{IMS}, is limited by the beam splitter (25% throughput) and the diffraction grating (80% diffraction efficiency) (FIG. 1), and η_{IMS}=20%. The quantum yield of the detector η_{D} is 95%. Therefore, the overall light throughput of the system was computed as η_{B}η_{IMS}η_{D}≈10%.

The signal-to-noise ratio (SNR) that can be expected with the system was estimated. Provided that the illumination intensity at the sample is 10 mW and the illuminated area is 10 mm in diameter, the illumination irradiance at the sample approximates 0.3 μW/mm²/nm, which is well below the ANSI safety standard (4 μW/mm²/nm). Next, the diffuse reflectance irradiance R_{d} was calculated using a Monte Carlo model based on typical tissue optical properties, and R_{d}≈30 nW/mm²/nm. Given 0.01 collection NA, the actual reflectance irradiance measured by the system is R′_{d}=0.1 nW/mm²/nm. Provided this irradiance was measured by the IMS operated at 50 fps, and the light energy contained in a hyperspectral datacube voxel (Δx=Δy=70 nm; Δλ=5 nm) is 4×10⁻⁵ nJ. Multiplying it with the system throughput yields the mapped light energy, E≈4×10⁻⁶ nJ, generating 13000 electrons at a detector pixel. Because the readout noise of sCMOS camera is low (1.3 electron rms), the system was considered to be shot-noise limited. Therefore, the expected SNR for a spectral channel image approximates 20 dB which was sufficient for HSI classification.

### Example 2

Because HSI generates a three-dimensional hyperspectral datacube that is generally large in size, a supervised deep-learning-based framework that is trained with the clinically visible tumor and benign tissue during surgery was developed. A deep convolutional neural network (CNN) was developed and compared with other types of classifiers.

A 2D-CNN architecture was constructed to include a modified version of the inception module appropriate for HSI that does not include max-pools and uses larger convolutional kernels, implemented using TensorFlow. The modified inception module simultaneously performs a series of convolutions with different kernel sizes: a 1×1 convolution; and convolutions with 3×3, 5×5, and 7×7 kernels following a 1×1 convolution.

The model consisted of two consecutive inception modules, followed by a traditional convolutional layer with a 9×9 kernel, followed by a final inception module. After the convolutional layers were two consecutive fully connected layers, followed by a final soft-max layer equal to the number of classes. A drop-out rate of 60% was applied after each layer. For binary classification, the number of convolutional filters were 355, 350, 75, and 350, and the fully connected layers had 256 and 218 neurons. For multi-class classification, the number of convolutional filters were 496, 464, 36, and 464, and the fully connected layers had 1024 and 512 neurons.

Convolutional units were activated using rectified linear units (ReLu) with Xavier convolutional initializer and a 0.1 constant initial neuron bias. Stepwise training was done in batches of 10 (for binary) or 15 (for multi-class) patches for each step. Every one-thousand steps the validation performance was evaluated, and the training data were randomly shuffled for improved training. Training was done using the AdaDelta, adaptive learning, optimizer for reducing the cross-entropy loss with an epsilon of 1×10⁻⁸ (for binary) or 1×10⁻⁹ (for multi-class) and rho of 0.8 (for binary) or 0.95 (for multi-class. For normal tissue versus cancer binary classification, the training was done at a learning rate of 0.05 for five to fifteen thousand steps depending on the patient-held-out iteration. For multi-class sub-classification of normal tissues, the training was done at a learning rate of 0.01 for three to five thousand steps depending on the patient-held-out iteration.

To test the classification accuracy of the models, 50 cancer patients who were undergoing surgical cancer resection were recruited and 88 excised tissue samples were collected. A convolutional neural network (CNN) was implemented using TensorFlow to classify the tissue as either normal or cancerous. The neural network architecture consisted of six convolutional layers and three fully connected layers. The output layer generated a probability of the pixel belonging to either class. Finally, the probability map was binarized to provide diagnostic cancer visualization.

For training and testing the CNN, each patient HSI was divided into patches. Patches were produced from each HSI after normalization and glare removal to create 25×25×91 non-overlapping patches that did not include any “black-holes” where pixels had been removed due to specular glare. Glare pixels were intentionally removed from the training dataset to avoid learning from impure samples. In addition, patches were augmented by 90-, 180-, and 270-degree rotations and vertical and horizontal reflections, to produce six times the number of samples. For cancer classification, the patches were extracted from the whole tissue. While for multi-class sub-classification of normal tissues, the regions of interest comprised of the classes of target tissue were extracted using the outlined gold-standard histopathology images.

The convolutional neural networks were built from scratch using the TensorFlow application program interface (API) for Python. A high-performance computer was used for running the experiments, operating on Linux Ubuntu 16.04 with 2 Intel Xeon 2.6 GHz processors, 512 GB of RAM, and 8 NVIDIA GeForce Titan XP GPUs. Two distinct CNN architectures were implemented for classification. During the following experiments, only the learning-related hyper-parameters that were adjusted between experiments, which include learning rate, decay of the AdaDelta gradient optimizer, and batch-size. Within each experiment type, the same learning rate, rho, and epsilon were used, but some cross-validation iterations used different numbers of training steps because of earlier or later training convergence.

The performance of the CNN was then evaluated with a cross-validation method. Histological images evaluated by a pathologist were used as a gold standard. Patient samples that are known to be of one class were used for the CNN training, and then new tissue was classified from that same patient for validation. This technique could augment the performance of the classification when a surgeon can provide a sample from the patient for training. The CNN was fully trained for 20,000 steps using the training dataset, and the performance was calculated on the testing dataset. Additionally, the performance of CNN was compared against several other classifiers, support vector machine (SVM), k-nearest neighbors (KNN), logistic regression (LR), complex decision tree classifier (DTC), and linear discriminant analysis (LDA). The results showed that CNN outperforms all other machine learning methods.

### Example 3

The imaging performance of the probe was initially characterized using optical phantoms to evaluate the system imaging. Initially, the spatial and spectral imaging performance of the system were characterized using standard targets. To characterize the system's spatial resolution, a USAF resolution target was imaged and then the resolution was calculated using a slanted-edge method. To measure the system's spectral resolution, a Lambertian-reflectance surface illuminated by monochromatic light was imaged and the spectra was averaged with the FOV and calculate the spectral resolution as the full-width-half-maximum of the correspondent spectral peak. Additionally, to quantify the spectral measurement accuracy, a standard color checker plate with a pattern of 24 scientifically prepared color squares (Edmund Optics) were imaged in sequence. The outcome of each measurement was an average spectrum over all pixels within a color square. To provide ground truth, the spectra was also measured using a benchmark spectrometer (Torus, Ocean Optics).

Then the spectra measured by the probe and Ocean Optics spectrometer was normalized for each color square. Next, the accuracy was quantified by calculating the RMSE of their spectral difference, RMSE=\sqrt{Σ_{λ}[S_{H}(λ)−S_{O}(λ)]2/M}, where S_{H}(λ) and S_{O}(λ) are the normalized spectra measured by the HSI probe and Ocean Optics spectrometer, respectively, and M is the total number of spectral channels. If the mean RMSEs for all color squares is no greater than 5%, the method was considered to be a success.

The ability of the system to classify objects based on measured spectra was tested on tissue-mimicking optical phantoms. The goal of phantom imaging was to fine tune the system and classification procedure to prepare for animal and human studies. The phantom may comprise two compartments filled with materials of different optical properties and separated by predefined boundaries. The phantom was made using gelatin gel uniformly mixed with intralipids as the scattering contrast and different color dyes as the absorption contrast for the two compartments. Therefore, the materials in the two compartments will exhibit different absorption spectra, mimicking the normal tissue and tumor. An en-face image could be captured on the hyperspectral datacube and classifications were performed. The recovered boundaries between the two compartments were then overlaid with the ground truth on the same image for comparison.

### Example 4

To further characterize the imaging performance of the probe, the system was evaluated using a porcine cancer model in vivo and with excised surgical tissue specimens. A transgenic porcine model, the Oncopig Cancer Model (OCM), was developed as a translational large animal platform for testing the cancer diagnostic, therapeutic, and imaging modalities. The OCM is a unique genotypically, anatomically, metabolically, and physiologically relevant large animal model for preclinical study of human cancer that develops inducible site/cell specific tumors. The OCM was designed to harbor heterozygous mutations found in >50% of human cancers: KRAS^{G12D} and TP53^{R167H} and results in tumors that recapitulate the phenotype and physiology of human cancers. As in human disease, TERT is solely expressed in OCM cancer cells, and innate OCM KRAS^{G12D} and TP53^{R167H} driver mutations are heterozygous in nature. OCM tumor development also occurs within a 1-month to 6-month time frame, which aligns well with the clinical disease course. Using the OCM, an Oncopig hepatocellular carcinoma (HCC) model was previously developed that recapitulates human disease, supporting the concept that mechanisms underlying OCM cancers provide insight into behaviors observed clinically in human cancers.

The OCM is an ideal model for the investigation of novel devices. Because the size and anatomy of pig lungs are similar to humans, the OCM provides the ability to perform bronchoscope-based imaging procedures using the same tools and techniques used in clinical practice.

An Oncopig lung cancer model was recently developed via intra-tracheal exposure to 1×10¹⁰ plaque forming units (PFU) of adenoviral vector encoding Cre recombinase and GFP (AdCre) suspended in 5 ml of PBS in 8-week-old Oncopigs. Two weeks post inoculation, a nodule measuring 1 cm in diameter was visible via CT. Following the CT scan, the Oncopig was euthanized, and the grossly visible mass was collected for histological evaluation. H&E staining was performed, and a proliferative lesion with regions of inflammation was identified by a human pathologist with expertise in lung cancer diagnostics. Expression of the KRAS^{G12D} mutation was confirmed via immunohistochemistry, confirming the observed lung tumor was the result of cellular transformation stemming from activation of the transgenes. The Oncopig lung tumors were classified based on clinically employed markers for diagnosis of human lung cancer subtypes.

The probe was then tested in vivo by imaging pig lung tumors induced using the Oncopig Cancer Model, a transgenic pig model that recapitulates human cancer through induced expression of heterozygous KRAS^{G12D} and TP53^{R167H} driver mutations.

Lung tumors were induced in Oncopigs (n=20; 10 male and 10 female) at an age of between 2 months and 6 months which is the age at which the pig airways are large enough to accommodate the bronchoscope for testing. In a surgical suite under general anesthesia, an endotracheal tube was placed in the trachea using the lighted guide of a laryngoscope. Oncopigs were inoculated with 5 ml of 1×10¹⁰ PFU of AdCre delivered through the endotracheal tube, which resulted in tumor formation within 2 weeks.

Following lung tumor induction, the Oncopigs entered an active surveillance program to assess for tumor growth. Contrast-enhanced CT was performed weekly following standard human lung CT protocols. CT images were used to identify the approximate size and location of lung tumors. Once identified, bronchoscope procedures were performed.

The probe and deep learning architecture were used to perform training and validation on the same Oncopig, and a sample of 20 Oncopigs (n=20; 10 male and 10 female) were imaged. In a surgical suite under general anesthesia, the bronchoscope can be placed into the airway of the pig and navigated to the tumor site. After the identification of clinically visible endobronchial lesions endoscopically, corresponding hyperspectral images were captured with the probe. Using endobronchial forceps through the working channel of the bronchoscope, under direct visualization, tumor biopsies were obtained. The biopsy samples were then H&E stained and imaged under a wide-field microscope to provide the ground truth diagnosis. Hyperspectral images and biopsies at benign tissue sites were collected following this procedure. The bronchoscope was withdrawn from the airway once adequate tissue and images were obtained.

The outcome measure at each imaging site was a hyperspectral datacube of dimension 150×150×100 (x, y, λ). The datacube was processed as outlined in FIG. 4 and extracted feature vectors of dimension 400 (1^{st} order derivatives of spectral curve; 2^{nd} order derivatives of spectral curve; mean, std, and total reflectance; Fourier coefficients) were obtained. The feature set that best characterize the difference between tumor and benign tissue was selected and the optimal feature dimension approximates 20.

Training data was also obtained. Provided that each training image contain only tumor or normal tissue within the FOV (150×150 pixels), the associated hyperspectral measurement contributed to 22,500 classified spatial-spectral feature vectors for each pair of tissue groups (tumor and benign).

Spatio-spectral changes between tumor and benign tissue were retained in the training model. This supported the expectation that tumor margin assessment based upon spatial and spectral information would be superior to predictions based on structural image only.

The development of the model in training was evaluated to assess how well the model can be used to accurately analyze tumor margins in vivo. Like the procedure in the training stage, clinically visible endobronchial lesions were identified through the bronchoscope. Then a hyperspectral reflectance image in a FOV that contains both the tumor and adjacent benign tissue was captured.

The reflectance spectra of the lung tumor and benign tissue in vivo were examined. The measured reflectance spectra associated with a clinically visible tumor was compared with the benign tissue through a MANOVA test. It had been previously discovered that the reflectance spectra of tumor and benign tissue significantly differ in head & neck surgical samples and similar spectral differences from in-vivo pulmonary tissue were expected as well. Due to the existence of blood, the in-vivo spectral signatures of the tumor and benign tissue were expected to be different from what they appear ex vivo. The comparison set the foundation for the in-vivo spectral classification.

The results showed that the white-light reflectance spectrum of the tumor is significantly different from that of normal tissue. By processing HSI datacubes using a machine-learning-based algorithm with a k-nearest neighbors (KNN) classifier, it was demonstrated that HSI was able to distinguish cancer from normal tissue, matching well with the ground truth.

### Example 5

To test HSI for cancer imaging in humans, surgical tissue specimens were collected from 16 human patients who underwent head-and-neck cancer surgery and imaged these in-vitro samples using a benchtop wavelength-scanning system. We used the spectra from 450 nm to 900 nm to extract the diagnostic information. For quantitative comparison, autofluorescence images and fluorescence images labeled with 2-NBDG and proflavine were also captured from each specimen. The post-imaging samples were Hematoxylin and Eosin (H&E) stained and examined by a pathologist to provide the ex-vivo ground truth.

The results show that the white-light reflectance spectrum of the tumor is significantly different from that of normal tissue. By processing HSI datacubes using a machine-learning-based algorithm with a k-nearest neighbors (KNN) classifier, it was demonstrated that HSI was able to distinguish cancer from normal tissue, matching well with the ground truth.

To quantify the classification results, the accuracy, sensitivity, and specification were calculated for all the lesions imaged (oral cavity, thyroid). Moreover, for the same specimen, the metrics were also computed based on the autofluorescence and fluorescence data. The results show that HSI outperformed autofluorescence imaging and fluorescence imaging in all evaluation metrics and confirmed the feasibility of label-free HSI for tumor margin assessment in surgical tissue specimens of cancer patients.

The snapshot hyperspectral imaging of human tissue in vivo by image mapping spectrometry was also evaluated. A prototype IMS was created and tested on human tissue in vivo. Tissue vascularization of the lower lip of a normal volunteer was initially evaluated with the IMS system. The tissue site was obliquely illuminated with a halogen lamp and the reflectance light was collected using a miniature objective lens and the image was then coupled into the distal end of an image fiber bundle. Next, the image was guided through the image fiber bundle to the proximal end to the input plane of the IMS. Within a single snapshot, the IMS could capture 29 spectral channels in the visible spectral range (450-650 nm). The frame rate was limited by the readout speed of the CCD camera in the IMS prototype and was up to 5 fps.

To further analyze the spectral signature of endogenous chromophores, spectral curves from two regions within the datacube were extracted. Lines were taken from a region in the datacube where there was a vein, and other lines taken from another region in the datacube where there was no vein. Dominating features in these spectral curves were successfully recovered that correspond to absorption peaks of oxyhemoglobin at 542 nm and 576 nm. Based on this spectral fingerprint, it was possible to enhance the contrast of the vasculature and obtain an image like that produced by angiography, but without the use of dyes.

Because the method can differentiate tumor and normal tissue in vivo without administering contrast agents to humans, the methods allow the surgeon to accurately localize and resect lung tumors while preserving healthy lung function.

Moreover, beyond lung cancer, the real-time HSI endoscopy system can also be used for imaging other malignant lesions, such as brain cancer, oral cancer, and colon cancer. Like lung cancer, their progression is often accompanied by abnormal structural and molecular changes, which can be inferred from HSI measurement. Delineating the tumor margins based on the spectral signatures can dramatically improve the safety and accuracy of surgical resection in these cancers as well.

Embodiments of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure (s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, that the terms processor, hardware processor, computer processor, central processing unit (CPU), and computer are used synonymously to denote a device capable of executing the instructions and communicating with input/output interfaces and/or peripheral devices, and that the terms processor, hardware processor, computer processor, CPU, and computer are intended to encompass single or multiple devices, single core and multicore devices, and variations thereof.

From the description herein, it will be appreciated that the present disclosure encompasses multiple implementations which include, but are not limited to, the following:

An apparatus for snapshot hyperspectral imaging, the apparatus comprising: (a) an endoscope with a light source configured to project a light to a target and an image fiber bundle and lens positioned at a distal end of the endoscope to receive reflected light from the target; and (b) an imager coupled to the image fiber bundle of the endoscope, the imager comprising: (i) a gradient-index lens optically coupled to the image fiber bundle of the endoscope and to an objective lens and tube lens; (ii) an image mapper; (iii) a collector lens; (iv) a diffraction grating or prism; (v) a reimaging lenslet array; and (vi) a light detector.

The apparatus of any preceding or following implementation, wherein the light source comprises a broadband light source, an endoscope illumination channel, and a light guide.

The apparatus of any preceding or following implementation, further comprising: a spatial filter positioned between the objective lens and the tube lens configured to remove a fiber bundle obscuration pattern.

The apparatus of any preceding or following implementation, wherein the image mapper comprises: a faceted mirror, the facets having a width, a length and a 2D tilt angle in an x-direction or a y-direction; wherein, light rays reflected from different mirror facets are collected by the collection lens.

The apparatus of any preceding or following implementation, further comprising: (a) a processor configured to control the light detector; and (b) a non-transitory memory storing instructions executable by the processor; (c) wherein the instructions, when executed by the processor, perform steps comprising: (i) forming hyperspectral data cubes from hyperspectral measurements of the light detector; (ii) pre-processing the datacubes to reduce dataset size; (iii) extracting spectral features from pre-processed data; (iv) selecting features that characterize differences between tumor and benign tissue; and (v) classifying tissue as tumor or benign.

The apparatus of any preceding or following implementation, wherein the formation of hyperspectral data cubes comprises: reverse mapping raw detector data to transform it into a datacube; normalizing an intensity response of every datacube voxel; and correcting for spectral sensitivity to produce an input hyperspectral datacube.

The apparatus of any preceding or following implementation, wherein the pre-processing comprises: removing hyperspectral data associated with glare pixels from analysis; normalizing spectral data; and correcting curvature to compensate for spectral variations caused by elevations in target tissue.

The apparatus of any preceding or following implementation, wherein the feature extraction comprises: applying a first-order derivative to each spectral curve to quantify the variations of spectral information across a wavelength range; applying a second-order derivative to each spectral curve to quantify the concavity of the spectral curve; calculating a mean standard deviation and total reflectance at each pixel; and calculating Fourier coefficients (FCs) for each feature is standardized to its z-score by subtracting the mean from each feature and then dividing by its standard deviation.

The apparatus of any preceding or following implementation, wherein said instructions when executed by the processor further perform steps comprising: training a Convolution Neural Network (CNN) on plurality of tumor and benign tissue spectral data to generate a classifier; and applying the classifier to newly formed hyperspectral data cubes to classify tissue as tumor or benign.

A method for hyperspectral imaging (HSI) endoscopy, the method comprising: (a) acquiring reflectance spectra from a target illuminated with white light; (b) forming one or more hyperspectral datacubes from the acquired reflectance spectra; (c) pre-processing the datacubes to reduce dataset size; (d) extracting spectral features from pre-processed data; (e) selecting features that characterize differences between tumor and benign tissue; and (f) classifying tissue as tumor or benign.

The method of any preceding or following implementation, wherein the formation of hyperspectral data cubes comprises: reverse mapping raw detector data to transform it into a datacube; normalizing an intensity response of every datacube voxel; and correcting for spectral sensitivity to produce an input hyperspectral datacube.

The method of any preceding or following implementation, wherein the pre-processing comprises: removing hyperspectral data associated with glare pixels from analysis; normalizing spectral data; and correcting curvature to compensate for spectral variations caused by elevations in target tissue.

The method of any preceding or following implementation, wherein the feature extraction comprises: applying a first-order derivative to each spectral curve to quantify the variations of spectral information across a wavelength range; applying a second-order derivative to each spectral curve to quantify the concavity of the spectral curve; calculating a mean standard deviation and total reflectance at each pixel; and calculating Fourier coefficients (FCs) for each feature is standardized to its z-score by subtracting the mean from each feature and then dividing by its standard deviation.

The method of any preceding or following implementation, further comprising: training a Convolution Neural Network (CNN) on plurality of tumor and benign tissue spectral data to generate a classifier; and applying the classifier to newly formed hyperspectral data cubes to classify tissue as tumor or benign.

The method of any preceding or following implementation, further comprising: selecting a classifier from the group of classifiers consisting of support vector machine (SVM), k-nearest neighbors (KNN), logistic regression (LR), complex decision tree classifier (DTC), and linear discriminant analysis (LDA); training the classifier on a plurality of tumor and benign tissue spectral data; and applying the classifier to newly formed hyperspectral data cubes to classify tissue as tumor or benign.

An apparatus for snapshot hyperspectral imaging, the apparatus comprising: (a) an endoscope with a light source configured to project a light to a target and an image fiber bundle and lens positioned at a distal end of the endoscope to receive reflected light from the target; and (b) an imager coupled to the image fiber bundle of the endoscope, the imager comprising: (i) a gradient-index lens optically coupled to the image fiber bundle of the endoscope and to an objective lens and tube lens; (ii) an image mapper; (iii) a collector lens; (iv) a diffraction grating or prism; (v) a reimaging lenslet array; and (vi) a light detector; (c) a processor configured to control the light detector; and (d) a non-transitory memory storing instructions executable by the processor; (e) wherein the instructions, when executed by the processor, perform steps comprising: (i) forming hyperspectral data cubes from hyperspectral measurements of the light detector; (ii) pre-processing the datacubes to reduce dataset size; (iii) extracting spectral features pre-processed data; (iv) selecting features that characterize differences between tumor and benign tissue; and (v) classifying tissue as tumor or benign.

The apparatus of any preceding or following implementation, wherein the formation of hyperspectral data cubes comprises: reverse mapping raw detector data to transform it into a datacube; normalizing an intensity response of every datacube voxel; and correcting for spectral sensitivity to produce an input hyperspectral datacube.

The apparatus of any preceding or following implementation, wherein the pre-processing comprises: removing hyperspectral data associated with glare pixels from analysis; normalizing spectral data; and correcting curvature to compensate for spectral variations caused by elevations in target tissue.

The apparatus of any preceding or following implementation, wherein the feature extraction comprises: applying a first-order derivative to each spectral curve to quantify the variations of spectral information across a wavelength range; applying a second-order derivative to each spectral curve to quantify the concavity of the spectral curve; calculating a mean standard deviation and total reflectance at each pixel; and calculating Fourier coefficients (FCs) for each feature is standardized to its z-score by subtracting the mean from each feature and then dividing by its standard deviation.

The apparatus of any preceding or following implementation, the instructions when executed by the processor further perform steps comprising: selecting a classifier from the group of classifiers consisting of support vector machine (SVM), k-nearest neighbors (KNN), logistic regression (LR), complex decision tree classifier (DTC), Convolution Neural Network (CNN) and linear discriminant analysis (LDA); training the classifier on a plurality of tumor and benign tissue spectral data; and applying the classifier to newly formed hyperspectral data cubes to classify tissue as tumor or benign.

As used herein, term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system, or method.

As used herein, the term “set” refers to a collection of one or more objects. Thus, for example, a set of objects can include a single object or multiple objects.

Relational terms such as first and second, top and bottom, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential features or elements of the technology describes herein or any or all the claims.

In addition, in the foregoing disclosure various features may grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after that application is filed. Accordingly the reader should consult the application as filed for the original content of the disclosure. Any deletion of content of the disclosure should not be construed as a disclaimer, forfeiture, or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An apparatus for snapshot hyperspectral imaging, the apparatus comprising:
(a) an endoscope with a light source configured to project a light to a target and an image fiber bundle and lens positioned at a distal end of the endoscope to receive reflected light from the target; and
(b) an imager coupled to the image fiber bundle of the endoscope, the imager comprising:
(i) a gradient-index lens optically coupled to the image fiber bundle of the endoscope and to an objective lens and tube lens;
(ii) an image mapper;
(iii) a collector lens;
(iv) a diffraction grating or prism;
(v) a reimaging lenslet array; and
(vi) a light detector.

2. The apparatus of claim 1, wherein said light source comprises a broadband light source, an endoscope illumination channel, and a light guide.

3. The apparatus of claim 1, further comprising:
a spatial filter positioned between the objective lens and the tube lens configured to remove a fiber bundle obscuration pattern.

4. The apparatus of claim 1, wherein said image mapper comprises:
a faceted mirror, said facets having a width, a length and a 2D tilt angle in an x-direction or a y-direction;
wherein, light rays reflected from different mirror facets are collected by the collection lens.

5. The apparatus of claim 1, further comprising:
(a) a processor configured to control said light detector; and
(b) a non-transitory memory storing instructions executable by the processor;
(c) wherein said instructions, when executed by the processor, perform steps comprising:
(i) forming hyperspectral data cubes from hyperspectral measurements of said light detector;
(ii) pre-processing the datacubes to reduce dataset size;
(iii) extracting spectral features from pre-processed data;
(iv) selecting features that characterize differences between tumor and benign tissue; and
(v) classifying tissue as tumor or benign.

6. The apparatus of claim 5, wherein said formation of hyperspectral data cubes comprises:
reverse mapping raw detector data to transform it into a datacube;
normalizing an intensity response of every datacube voxel; and
correcting for spectral sensitivity to produce an input hyperspectral datacube.

7. The apparatus of claim 5, wherein said pre-processing comprises:
removing hyperspectral data associated with glare pixels from analysis;
normalizing spectral data; and
correcting curvature to compensate for spectral variations caused by elevations in target tissue.

8. The apparatus of claim 5, wherein said feature extraction comprises:
applying a first-order derivative to each spectral curve to quantify the variations of spectral information across a wavelength range;
applying a second-order derivative to each spectral curve to quantify the concavity of the spectral curve;
calculating a mean standard deviation and total reflectance at each pixel; and
calculating Fourier coefficients (FCs) for each feature is standardized to its z-score by subtracting the mean from each feature and then dividing by its standard deviation.

9. The apparatus of claim 5, wherein said instructions when executed by the processor further perform steps comprising:
training a Convolution Neural Network (CNN) on plurality of tumor and benign tissue spectral data to generate a classifier; and
applying the classifier to newly formed hyperspectral data cubes to classify tissue as tumor or benign.

10. A method for hyperspectral imaging (HSI) endoscopy, the method comprising:
(a) acquiring reflectance spectra from a target illuminated with white light;
(b) forming one or more hyperspectral datacubes from the acquired reflectance spectra;
(c) pre-processing the datacubes to reduce dataset size;
(d) extracting spectral features from pre-processed data;
(e) selecting features that characterize differences between tumor and benign tissue; and
(f) classifying tissue as tumor or benign.

11. The method of claim 10, wherein said formation of hyperspectral data cubes comprises:
reverse mapping raw detector data to transform it into a datacube;
normalizing an intensity response of every datacube voxel; and
correcting for spectral sensitivity to produce an input hyperspectral datacube.

12. The method of claim 10, wherein said pre-processing comprises:
removing hyperspectral data associated with glare pixels from analysis;
normalizing spectral data; and
correcting curvature to compensate for spectral variations caused by elevations in target tissue.

13. The method of claim 10, wherein said feature extraction comprises:
applying a first-order derivative to each spectral curve to quantify the variations of spectral information across a wavelength range;
applying a second-order derivative to each spectral curve to quantify the concavity of the spectral curve;
calculating a mean standard deviation and total reflectance at each pixel; and
calculating Fourier coefficients (FCs) for each feature is standardized to its z-score by subtracting the mean from each feature and then dividing by its standard deviation.

14. The method of claim 10, further comprising:
training a Convolution Neural Network (CNN) on plurality of tumor and benign tissue spectral data to generate a classifier; and
applying the classifier to newly formed hyperspectral data cubes to classify tissue as tumor or benign.

15. The method of claim 10, further comprising:
selecting a classifier from the group of classifiers consisting of support vector machine (SVM), k-nearest neighbors (KNN), logistic regression (LR), complex decision tree classifier (DTC), and linear discriminant analysis (LDA);
training the classifier on a plurality of tumor and benign tissue spectral data; and
applying the classifier to newly formed hyperspectral data cubes to classify tissue as tumor or benign.

16. An apparatus for snapshot hyperspectral imaging, the apparatus comprising:
(a) an endoscope with a light source configured to project a light to a target and an image fiber bundle and lens positioned at a distal end of the endoscope to receive reflected light from the target; and
(b) an imager coupled to the image fiber bundle of the endoscope, the imager comprising:
(i) a gradient-index lens optically coupled to the image fiber bundle of the endoscope and to an objective lens and tube lens;
(ii) an image mapper;
(iii) a collector lens;
(iv) a diffraction grating or prism;
(v) a reimaging lenslet array; and
(vi) a light detector;
(c) a processor configured to control said light detector; and
(d) a non-transitory memory storing instructions executable by the processor;
(e) wherein said instructions, when executed by the processor, perform steps comprising:
(i) forming hyperspectral data cubes from hyperspectral measurements of said light detector;
(ii) pre-processing the datacubes to reduce dataset size;
(iii) extracting spectral features pre-processed data;
(iv) selecting features that characterize differences between tumor and benign tissue; and
(v) classifying tissue as tumor or benign.

17. The apparatus of claim 16, wherein said formation of hyperspectral data cubes comprises:
reverse mapping raw detector data to transform it into a datacube;
normalizing an intensity response of every datacube voxel; and
correcting for spectral sensitivity to produce an input hyperspectral datacube.

18. The apparatus of claim 16, wherein said pre-processing comprises:
removing hyperspectral data associated with glare pixels from analysis;
normalizing spectral data; and
correcting curvature to compensate for spectral variations caused by elevations in target tissue.

19. The apparatus of claim 16, wherein said feature extraction comprises:
applying a first-order derivative to each spectral curve to quantify the variations of spectral information across a wavelength range;
applying a second-order derivative to each spectral curve to quantify the concavity of the spectral curve;
calculating a mean standard deviation and total reflectance at each pixel; and
calculating Fourier coefficients (FCs) for each feature is standardized to its z-score by subtracting the mean from each feature and then dividing by its standard deviation.

20. The apparatus of claim 16, wherein said instructions when executed by the processor further perform steps comprising:
selecting a classifier from the group of classifiers consisting of support vector machine (SVM), k-nearest neighbors (KNN), logistic regression (LR), complex decision tree classifier (DTC), Convolution Neural Network (CNN) and linear discriminant analysis (LDA);
training the classifier on a plurality of tumor and benign tissue spectral data; and
applying the classifier to newly formed hyperspectral data cubes to classify tissue as tumor or benign.
